(19) United States
(12) Patent Application Publication
(10) Pub. No.: US 2018/0303542 A1
(43) Pub. Date: Oct. 25, 2018

(54) RF Energy Delivery Device for Vaginal Tightening

(71) Applicant: Eclipse Aesthetics, LLC, The Colony, TX (US)

(72) Inventor: Thomas Michael O'Brien, III, Dallas, TX (US)

(21) Appl. No.: 15/782,850

(22) Filed: Oct. 13, 2017

Related U.S. Application Data
(60) Provisional application No. 62/408,150, filed on Oct. 14, 2016.
(60) Provisional application No. 62/419,129, filed on Nov. 8, 2016.

(51) Int. Cl. A61B 18/14; A61N 1/40

(52) U.S. Cl. CPC A61B 18/1485; A61N 2005/0611; A61N 1/403

(57) ABSTRACT

Disclosed is a system and related method for delivering RF energy to a vaginal cavity for vaginal rejuvenation. The RF energy delivery system may comprise an RF energy delivery device, further comprising a plurality of RF electrodes, an RF oscillator for generating RF power, an RF amplifier connected to the RF oscillator, a programmable controller; and a power source. The RF oscillator may be configured to generate an RF signal based on information sent from the programmable controller. The RF amplifier may receive the generated RF signal and may be configured to amplify the RF signal to an appropriate power level for the plurality of RF electrodes, and the plurality of RF electrodes may receive the amplified RF signal and generate RF energy from the amplified RF signal.

### CROSS-REFERENCE TO RELATED APPLICATION

This application claims the benefit of and priority to U.S. Provisional Patent Application No. 62/408,150 filed Oct. 14, 2016 and to U.S. Provisional Patent Application No. 62/419,129 filed on Nov. 8, 2016, the technical disclosures of which is hereby incorporated herein by reference.

### BACKGROUND OF THE INVENTION

### Technical Field

This present invention relates to an apparatus for the delivery of RF energy to the vagina and vaginal areas for the purpose of tightening and rejuvenating vaginal tissues, and a method for delivering RF energy to the vaginal areas.

### Description of Related Art

Radiofrequency (RF) energy is a form of electromagnetic energy that is a part of the electromagnetic spectrum, which includes visible light, microwaves, and X-rays. RF energy, which may also be called RF emissions, RF waves, or RF fields, is generated when electric currents oscillate at radio frequencies (ranging from around 3 kHz to 300 GHz). The RF current runs along the surface of any electrical conductor, and when the human body comes into contact with high power RF currents, the RF currents can cause superficial but serious burns. However, in lower degrees, RF currents do not cause the painful sensation of electric shock or burns, because the RF current changes direction too quickly to depolarize nerve membranes, which leads to electric shock.

RF energy, in the form of radiating waves or electrical currents, has been used in the medicine for minimally invasive surgeries using radiofrequency ablation. RF energy is known for treating sleep apnea, pain, urinary symptoms, and other medical conditions. RF energy at non-ablation levels are also used cosmetically to tighten skin, reduce fat, and promote healing.

Currently, dermal tissues are exposed to RF energy in order to heat the tissues by four (4) to eight (8) degrees Celsius to trigger a healing response in the exposed dermal tissue. One device that uses RF energy on dermal tissues in the vaginal area is ThermiVa, a thermistor-regulated RF energy emission device used for non-surgical dermatological applications. The ThermiVa device draws RF current from an RF generator and the ThermiVa device generates RF energy to apply to vaginal tissues. However, the RF energy is focused near the tip of the ThermiVa device where the electrodes of the device are located, and the ThermiVa device requires the RF generator to generate the RF currents and RF energy.

U.S. Patent Application Publication No. US 2015/0366747, entitled Virginal Rejuvenation Apparatus and Method, provides another radio-frequency apparatus for vaginal tightening. This apparatus includes a main engine, a connection device, an applicator, and a negative plate. The applicator has a hand-held component and a treatment plug with multiple RF electrodes. This device also includes a built in cooling feature, in case the device overheats because of the heating caused by the RF energy. Furthermore, this device is designed for use at an office and not at home, given that the device requires a large office-sized RF generator.

The current state of the art of RF energy delivery to vaginal areas is limited. Current RF energy delivery devices require connection to RF generators that can only be found in medical offices or hospitals. Some devices also require integrated cooling units to prevent the RF energy from over-heating or damaging the internal electrical components. Further, some devices involve heating the vaginal cavity at high temperatures (over fifty degrees Celsius) in order to denature collagen, and cooling the vaginal cavity (before and/or after the heating) as a part of the treatment regime. Consequently, these devices require large, office-based RF generators in order to achieve the high and low temperatures for this treatment regime. There is a need for a system, device, or method for RF energy delivery that incorporates the RF generator and does not require any cooling system.

### SUMMARY

To overcome the deficiencies in the prior art, the disclosed principles provide for various embodiments of RF energy delivery devices for vaginal tightening and rejuvenation.

In accordance with a first embodiment, an RF delivery system for vaginal tightening and rejuvenation is provided. The RF energy delivery system may comprise an RF energy delivery device, further comprising a plurality of RF electrodes, an RF oscillator for generating RF power; an RF amplifier connected to the RF oscillator; a programmable controller; and a power source. The RF oscillator may be configured to generate an RF signal based on information sent from the programmable controller, and then the RF amplifier may receive the generated RF signal and may be configured to amplify the RF signal to an appropriate power level for the plurality of RF electrodes. Then, the plurality of RF electrodes may receive the amplified RF signal and generate RF energy from the amplified RF signal.

Optionally, the power source may be a battery or DC power. In another embodiment, the frequency of the generated RF signal may be equal to or greater than 1 MHz, and in yet another embodiment, the frequency of the generated RF signal is equal to or less than 6 MHz. Additionally, the RF energy delivery device may be constructed from biocompatible materials, and the RF electrodes may comprise biocompatible materials.

In other embodiments, the system may further comprise a temperature feedback sensor configured to update the information sent to the RF oscillator based on temperature feedback data transmitted from the temperature feedback sensor. The delivery device of the system may also comprise a vibratory function.

In another aspect, a method for delivering RF energy for vaginal tightening and rejuvenation is also provided. In an exemplary embodiment, such a method may comprise inserting an RF energy delivery device into the vaginal cavity; generating an RF signal with properties determined by a programmable controller; amplifying the RF signal; and transmitting the amplified RF signal to a plurality of RF electrodes to generate RF energy. Furthermore, in such exemplary embodiments, a method may further provide for a temperature feedback sensor transmitting temperature data to the programmable controller and the programmable controller adjusting the properties for generating the RF signal.

In other embodiments, the system may include a method for either randomly or sequentially selecting which electrode(s) to energize, so as to localize therapy but minimize overall heating of the intra-vaginal tissue.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a system view of the RF energy delivery system.

### DETAILED DESCRIPTION

In view of the foregoing, through one or more various aspects, embodiments and/or specific features or sub-components, the present disclosure is thus intended to bring out one or more of the advantages that will be evident from the description. The present disclosure makes reference to one or more specific embodiments by way of illustration and example. It is understood, therefore, that the terminology, examples, drawings and embodiments are illustrative and are not intended to limit the scope of the disclosure.

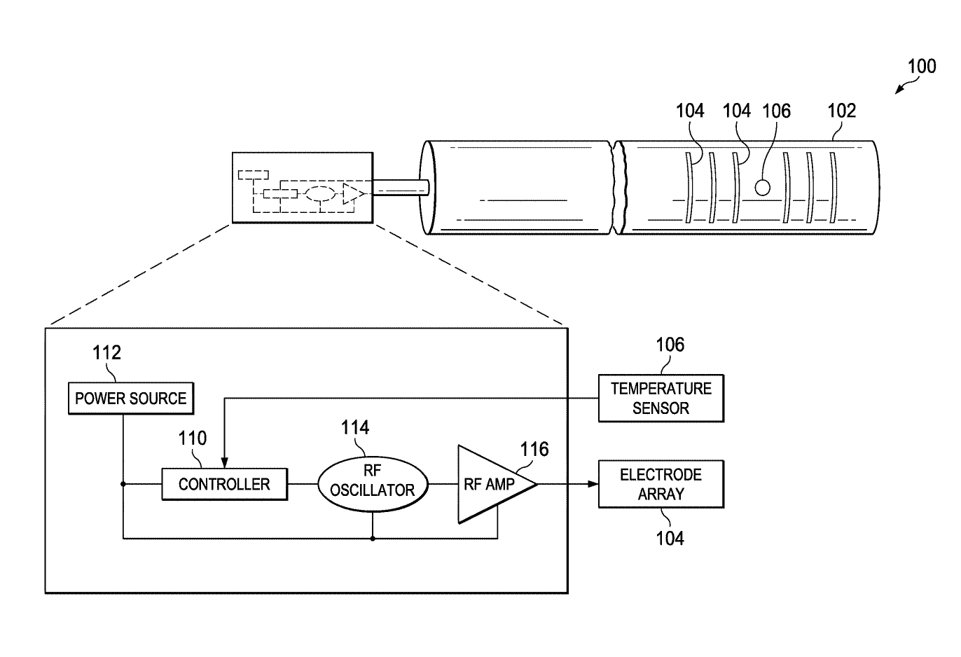

FIG. 1 is a system view of the RF energy delivery system 100. The RF energy delivery system 100 can be generally used for delivery RF energy to the vaginal cavity for the purpose of vaginal tightening and rejuvenation using an RF energy delivery device 102. The RF energy delivery device 102, which includes an RF electrode array 104 and a temperature feedback sensor 106, is connected to a programmable controller 110 and to an RF amplifier 116. The programmable controller 110 and RF amplifier 116 are both connected to an RF oscillator 114. A power source 112 delivers power to each component of the RF energy delivery system 100.

The RF energy delivery device 102 is housing for an RF electrode array 104 and is optimally shaped to match the shape of the vaginal canal and comprises an RF electrode array 104 and a temperature feedback sensor 106. The RF energy delivery device 102 may also house the other components of the RF energy delivery system 100, such as the programmable controller 110, the power source 112, the RF oscillator 114, and the RF amplifier 116. In one embodiment, the RF energy delivery device 102 is constructed from a semi-rigid material so that the RF energy delivery device 102 can mold itself to the shape of the vaginal cavity once inserted. The RF energy delivery device 102 must be made out biocompatible materials because the outside surface of the RF energy delivery device 102 is in direct contact with the walls of the vaginal cavity.

The RF electrode array 104 contacts the walls of the vaginal cavity and is mounted on the RF energy delivery device 102. The RF electrode array 104 may comprise one or more RF electrodes and may increase the temperature of the walls of the vaginal cavity by four to five degrees Celsius. The RF electrode array 104 may also be made of biocompatible materials because it is in direct contact with the vaginal cavity. The RF electrode array 104 delivers controlled RF energy to the vaginal cavity and surrounding areas. The RF electrode array 104 receives the RF energy via RF currents from the RF amplifier 116, and the programmable controller 110 monitors the RF energy and RF currents from the RF amplifier 116 and RF oscillator 114. The RF electrode array 104 may be arranged in any arrangement to effectively deliver RF energy to the vaginal cavity, and may be arranged to treat the full surface area of the vaginal cavity that is contacting the RF electrode array 104.

The RF energy delivery device 102 uses a temperature feedback sensor 106 on its surface to monitor the RF energy delivered to the vaginal cavity and to also monitor the temperature of the vaginal cavity. One of ordinary skill in the art will appreciate the construction, choice of type of temperature sensor, programming, operation, and functionality of such temperature sensor is well known, rendering further description of such devices unnecessary in this regard.

The programmable controller 110 automates the RF energy delivery system 100 and controls the function of the RF oscillator 114. The programmable controller 110 modulates the duty cycle of the RF oscillator 114 and therefore controls the RF energy output by the RF electrode array 104. The programmable controller 110 also monitors the power delivery to the system 100 by the power source 112. The programmable controller 110 may be connected to a pulse width modulator or a programmable digital control to ensure consistent output of the RF oscillator 114. The programmable controller 110 may incorporate any number of analog to digital converters and digital to analog converters for controlling either the power supply 112, the RF oscillator 114, or the RF amplifier 116. The controller 110 may also be connected to various input/output connectors for connecting to external data collection devices. The controller 110 may also comprise a processor and memory devices (or any other tangible non-transitory computer useable medium) for automating the RF energy delivery system 100.

The programmable controller 110 may be connected to a computer, smartphone, tablet, or any other computing device. By connecting the programmable controller 110 to a computer device, the RF energy delivery system 100 may be programmed to perform certain tasks, like those further disclosed below. A computing device may be any electrical device capable of accepting stored program instructions from a computer readable medium and processing those program instructions to perform a defined task. Such devices include, but are not limited to, a mainframe, workstation, desktop, laptop, notebook, or tablet computer, a database server, web server, or the like. The programmable controller 110 may comprise TI MSP 430 or Microchip IC 16 microcontrollers. One of ordinary skill in the art will appreciate that the construction, choice of programming language, programming, operation, and functionality of such computer processing devices is well known, rendering further description of such devices unnecessary in this regard.

The RF energy delivery system 100 may be extemporaneously programmed in an ad-hoc fashion based on information provided by a user. A user may program the controller 110 to automate the RF energy delivery system 100 in certain ways. For example, the user may program the RF energy delivery system 100 to deliver RF energy at a lower frequency as compared to the default rate, and so the rate of RF energy delivery into the vaginal cavity is also lower compared to the default rate of RF energy delivery. The programmable controller 110 may also be programmed to automate the RF energy delivery system 100 for a limited period of time. The RF electrode array 104 may also be programmed so that the user can direct RF current and RF energy through certain electrodes of the array 104.

The RF energy delivery of the system 100 is relatively low energy because the RF electrodes 104 heat the tissues of the vaginal cavity only one to five degrees Celsius. The system 100 only delivers enough energy to stimulate a healing cascade for generating collagen, while too much heating may cause a denaturing of the collagen. The system 100 is designed to treat the skin of the vaginal cavity, from the epidermis to the dermis, and the RF energy can be applied substantially into the vaginal cavity at significantly greater depths than current devices allow.

In alternative embodiments, the RF energy delivery system 100 may be programmed with a treatment regimen so that RF energy is delivered over a series of treatments. A treatment regimen with the RF energy delivery system 100 may change how frequently a patient needs to make visits to a doctor's office. Similar to how the programmable controller 110 may be programed to automate the RF energy delivery system at a lower frequency, the system 100 may be programmed to deliver RF energy at a lower temperature over a series of treatments (monthly, weekly, or daily) via the programmable controller 110. By lowering the temperature, the system 100 allows for a higher amount of comfort for the patient, and by delivering RF energy at a lower temperature over a series of treatments, the system 100 may allow for the approximately the same amount of efficacy as treating once at higher temperatures. Alternatively, the system 100 may also be programmed to deliver RF energy at a higher temperature over a smaller number of treatments or a shorter treatment time as compared to the series of treatments with a lower temperature. Delivering RF energy at a higher temperature over a smaller number of treatment or a shorter treatment time may reduce the time that the patient is exposed to RF energy and may reduce the overall treatment time.

The treatment regimen may also include energization of specific electrodes, randomly or sequentially, according to a preset program to treat tissue at a specific location, but to minimize overall heating and exposure to RF energy. Sequential energization of specific electrodes may include energizing electrodes in a certain direction, from one end of the electrode array 104 to the other end of the array 104. Random energization may include randomly selecting electrodes from the array 104 to energize.

In another embodiment, the RF energy delivery system 100 also includes a vibratory feature so as to distract the patient from any discomfort caused by the delivery of RF energy, especially any discomfort caused by heating the vaginal tissues. The vibratory feature may also distract the patient from any discomfort caused by the insertion of the RF energy delivery device 102 into the vaginal cavity. In order to include a vibratory feature, the system 100 may comprise a vibration-generating motor, which may be connected to the programmable controller 110 and the power source 112. The system 100 may be programmed to automate the vibration-generating motor for specific periods of times, for when requested by the client, and for other pre-programmed situations. The vibration-generating motor may also be automated with certain properties, such as different vibration frequencies, in order to provide patient comfort or distraction from any discomfort caused by RF energy delivery. One of ordinary skill in the art will appreciate the construction, choice of type of the vibration-generating motor, programming, and operation. Functionality of such vibration-generating motor is well known, rendering further description of such devices unnecessary in this regard.

In another embodiment, the RF energy delivery system 100 uses information data transmitted from the temperature feedback sensor 106 in the programming of the system 100. The temperature feedback sensor 106 delivers data to the programmable controller 110, such as the current temperature of the vaginal cavity, and responsive to the temperature reading, the programmable controller 110 may reduce the RF energy delivered to the vaginal cavity or pause the RF energy delivery process. The programmable controller 110, using the information from the temperature feedback sensor 106, may send appropriate signals to the RF oscillator 114 for reducing or pausing RF energy delivery.

The RF oscillator 114 is connected to the programmable controller 110 and generates the RF current and RF energy for the RF energy delivery system 100. The programmable controller 110 sends instructions to the RF oscillator 114 to generate an RF current or an RF signal with certain properties, such as a specific frequency, phase, amplitude, etc. The RF oscillator 114 may generate RF current based on the instructions from the programmable controller 110. The generated RF current may have oscillations ranging from 1 MHz to 6 MHz, and in other embodiments, the RF oscillator 114 may generate oscillating RF current greater than 6 MHz or less than 1 MHz. Op-amps, like an LM4562, may be used as an RF oscillator. One of ordinary skill in the art will appreciate the construction, choice of type of RF oscillator, programming, and operation, and functionality of such temperature sensor is well known, rendering further description of such devices unnecessary in this regard.

The RF amplifier 116 is connected to the RF oscillator 114 and transmits the generated RF current to the RF electrode array 106. The RF amplifier 116 takes in a generated RF signal or RF current from the RF oscillator 114 and amplifies the RF current or signal to an appropriate power level to drive the RF electrode array 106. Transistors, like any number of Infineon MOSFETs, may be used for the RF amplifier 116. One of ordinary skill in the art will appreciate the construction, choice of type of RF amplifier, programming, and operation, and functionality of such temperature sensor is well known, rendering further description of such devices unnecessary in this regard.

The power source 112 supplies power to the controller 110 and to the other components of the RF energy delivery system 100. The power supply 112 may be a battery built into the system, or an external power supply, such as an AC/DC adapter. One of ordinary skill in the art would know how to connect the power supply 112 to the controller 110.

The invention may be embodied in other specific forms without departing from the spirit or essential characteristics thereof. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the recitation of method steps does not denote a particular sequence for execution of the steps. Such method steps may therefore be performed in a sequence other than recited unless the particular claim expressly states otherwise.

1. A system for delivering RF energy to a vaginal cavity for vaginal rejuvenation, comprising:
an RF energy delivery device, further comprising a plurality of RF electrodes;
an RF oscillator for generating RF power;
an RF amplifier connected to the RF oscillator;
a programmable controller; and
a power source;
wherein
the RF oscillator is configured to generate an RF signal based on information sent from the programmable controller;
the RF amplifier receives the generated RF signal and is configured to amplify the RF signal to an appropriate power level for the plurality of RF electrodes; and
the plurality of RF electrodes receives the amplified RF signal and generates RF energy from the amplified RF signal.

2. The system of claim 1, wherein the power source is at least one of a battery and DC power.

3. The system of claim 1, wherein a frequency of the generated RF signal is equal to or greater than 1 MHz.

4. The system of claim 1, wherein a frequency of the generated RF signal is equal to or less than 6 MHz.

5. The system of claim 1, wherein the RF energy delivery device comprises biocompatible materials.

6. The system of claim 1, wherein the plurality of electrodes comprise biocompatible materials.

7. The system of claim 1, further comprising a temperature feedback sensor, wherein the programmable controller is configured to update the information sent to the RF oscillator based on temperature feedback data transmitted from the temperature feedback sensor.

8. The system of claim 1, further comprising a vibration-generating motor.

9. The system of claim 1, wherein the programmable controller is programmed to automate the system for a series of treatments.

10. A method for delivering RF energy to a vaginal cavity for vaginal rejuvenation, comprising:
inserting an RF energy delivery device into the vaginal cavity;
generating an RF signal with properties determined by a programmable controller;
amplifying the RF signal; and
transmitting the amplified RF signal to a plurality of RF electrodes to generate RF energy.

11. The method of claim 0, wherein a temperature feedback sensor transmits temperature data to the programmable controller and the programmable controller adjusts the properties for generating the RF signal.

12. The method of claim 0, wherein the RF energy delivery device comprises bio-compatible materials.

13. The method of claim 0, wherein the RF signal has a frequency equal to or greater than 1 MHz.

14. The method of claim 0, wherein the RF signal has a frequency equal to or less than 6 MHz.

15. The method of claim 0, wherein the plurality of electrodes comprise biocompatible materials.

16. The method of claim 0, further comprising activating a vibration-generating motor.

17. The method of claim 10, wherein the properties of the RF signal comprise a selection of RF electrodes from the plurality of RF electrodes to activate.
